(19) United States
(12) Patent Application Publication
(10) Pub. No.: US 2014/0018558 A1
(43) Pub. Date: Jan. 16, 2014

(54) DOCOSAHEXAENOIC ACID (DHA) AS POLYUNSATURATED FREE FATTY ACID IN ITS DIRECTLY COMPRESSIBLE POWDER FORM AND METHOD OF ISOLATION THEREOF

(71) Applicant: Nandakishore Jeevanrao Duragkar, Nagpur (IN)

(72) Inventor: Nandakishore Jeevanrao Duragkar, Nagpur (IN)

(21) Appl. No.: 14/007,013

(22) PCT Filed: Apr. 27, 2012
(86) PCT No.: PCT/IN12/00309

§ 371 Date: Sep. 24, 2013

(30) Foreign Application Priority Data
Apr. 28, 2011 (IN) 951/MUM/2011

(51) Int. Cl. C07C 51/09; C07C 57/03

(52) U.S. Cl. CPC C07C 51/09; C07C 57/03
USPC 554/8; 554/224

(57) ABSTRACT

The present invention provides Docosahexaenoic acid (DHA) in its free fatty acid form and a process of isolation thereof from oils and fats of natural origin having Docosahexaenoic acid (DHA) attached to triglycerides. The DHA isolated using the process of the present invention is in free flowing powder form which is directly compressible. Further, the DHA in powder form is free from triglycerides. The DHA powder of the present invention has purity more than 90%. The DHA in its free fatty acid powder form offers excellent bioavailability and stability at room temperature.

### FIELD OF INVENTION

The present invention relates to Docosahexaenoic acid (DHA) in its polyunsaturated free fatty acid, directly compressible powder form, and isolation of Docosahexaenoic acid (DHA) from natural sources of oil or fats having Docosahexaenoic acid (DHA) attached to triglycerides.

### BACKGROUND OF THE INVENTION

The extraction and isolation of important polyunsaturated fatty acid, Docosahexaenoic acid is done from fats and fatty oils available as triglycerides of fatty acids from their natural sources. Docosahexaenoic acid (DHA) is an omega-3 fatty acid that is a primary structural component of the human brain cerebral cortex, sperm, testicles and retina. Cold-water oceanic fish oils are rich in DHA. Most of the DHA in fish and multi-cellular organisms with access to cold-water oceanic foods originates from photosynthetic and heterotrophic microalgae, and becomes increasingly concentrated in organisms the further they are up the food chain. DHA is internally converted/synthesized from α-linolenic acid, a shorter omega-3 fatty acid. Dietary DHA may reduce the risk of heart disease by reducing the level of blood triglycerides in humans. Below-normal levels of DHA have been associated with Alzheimer's disease. A low level of DHA is also spotted in patients with retinitis pigmentosa. It is very difficult to obtain pure DHA in free fatty acid form as they are found usually as triglycerides in fats and oils of oceanic animals, fish oils (such as mackerel oil, menhaden oil, salmon oil, capelin oil, tuna oil, sardine oil, or cod oil), marine algae such as Schizochytrium sp., and as phospholipids in krill and also in human milk, and chicken head and egg yolk.

It is evident from previous literature searches that several attempts were made to isolate DHA from their triglycerides for producing highly pure free fatty acids. The previously attempted research resulted into the free fatty acids, which are in the liquid form. However, looking at the immense potential of this product in the commercial market, several attempts were made to incorporate these fatty acids in powder form by means of adsorption, encapsulation, spray drying the emulsion and also powder of egg yolks and chicken heads and direct drying of algae source of these fatty acids to get powder. The main concern in these cases were difficulty in achieving desired purity, desired levels of separation of fatty acids, stability of product and incorporation in the dry dosage forms and nutrition products along with limitations of compressibility. Therefore there was necessity to have DHA polyunsaturated free fatty acid in directly compressible form with an added advantage of stability at room temperature.

An U.S. Pat. No. 6,846,942 discloses a method for obtaining pure EPA and pure DHA from natural sources. These sources of DHA normally contains substantial amount of fatty acid residues, often as residues of triglyceride molecules, which dilutes the concentration of DHA in the oil. Other fatty acids are always present in larger amounts. The process in this patent involves saponification under controlled temperature of not exceeding 40° C., involves steps of purification which selectively led to a product which is a mixture of EPA and DHA in liquid form. It is very essential to add antioxidants at this stage to prevent oxidation of these fatty acids since in this form these fatty acids are highly unstable. Subsequently to separate EPA from DHA the magnesium salts of EPA & DHA are prepared which relies mostly on fractional precipitation using varying solubility of these fatty acids as salts in different solvents at subzero temperature.

An US Patent US 2008/0279935 A1 attempts to present DHA in a powder form. This form of DHA is an encapsulated DHA powder. Encapsulation of these fatty acids was a necessity for improving the handling properties of a liquid and sticky form fatty acids material. The material needs to be processed with so many components leading to dilution of fatty acids in these compositions. Moreover it does not offer fatty acids free from triglycerides. This material offered is in ester form and has almost five times less bioavailability. Since the material is microencapsulated, it cannot be directly compressible.

US Patent 2007/0059340 provides DHA food products, the process involves a Zinc coating to protect and stabilize the omega 3-fatty acids. These fatty acids are also in oil form and needs stabilization. However, it has all the disadvantages mentioned in the microencapsulation process.

The US Patent application 2010/0055191 discloses a method of providing DHA where a powder composition of a functional oil material is obtained by drying an emulsion composition and water soluble encapsulating agent. Due to unstable nature of DHA in oil form, efforts were made to stabilize DHA. However, it does not offer free flowing Pure polyunsaturated free fatty acid DHA and it has all the disadvantages mentioned in the microencapsulation process.

In US Patent No. 2011/0165233 additives were used to stabilize DHA when compressed into tablets, or filled as a powder into capsules, for oral administration. Further, WO/88/02221 patent application discloses a method of providing DHA granulates wherein PUFA containing lipid is adsorbed onto a solid carrier, such as powder it does not offer free flowing pure free fatty acid DHA. However, it has all the disadvantages mentioned in the microencapsulation process.

The DHA provided in prior art processes is either in form of liquid or liquid adsorbed on the powder. Any Such form of DHA till now cannot be used directly as a single constituent as polyunsaturated free fatty acid in directly compressible powder forms because of its inherent abovementioned problems. The available form of DHA renders it to be processed by some or other methods for getting its immense nutritional and curative benefits and therefore it was envisaged to offer a product which takes care of all the above mentioned problems through our process which is shorter, simpler and economical.

In our invention we get the highly pure DHA as polyunsaturated free fatty acid in free flowing powder form which is directly compressible and is substantially free from water. Being powder, it is found to be stable at room temperature and therefore, addition of antioxidants is not required. The process has minimum steps of purification and doesn't require subzero temperature at any stage for isolation of free fatty acid form of DHA. It is highly compatible and stable in any form of compositions like solid compositions, liquid compositions, powder compositions, tablets, capsules, gels and all other forms of formulations providing this polyunsaturated free fatty acid DHA.

The present invention seeks to provide a process of extraction and isolation of a pure polyunsaturated free fatty acid DHA from its natural sources in free flowing powder form which is directly compressible, that may solve all of the above mentioned problems in the prior art.

### OBJECTS OF THE INVENTION

An object of the present invention is to offer a unique form of the polyunsaturated free fatty acid in a directly compressible powder form (Solid) which is in its free fatty acid form.

Another object of the present invention is also to avoid the deficiencies in the prior art.

Yet another object of the present invention is also to provide pure DHA in free flowing powder form, which is directly compressible.

Another object of the present invention is to provide pure DHA as polyunsaturated free fatty acid form which is free from triglycerides.

Yet another object of the present invention is to provide DHA powder with purity of more than 90%.

Another object of the present invention is to provide DHA which is stable at room temperature.

One more object of the present invention is to provide DHA which offers excellent bioavailability.

### SUMMARY OF THE INVENTION

Accordingly, the present invention provides docosahexaenoic acid (DHA) as a polyunsaturated free fatty acid in a free flowing directly compressible powder form and method of isolation of docosahexaenoic acid (DHA), and the method comprising; Selecting any one of oil or fats from natural sources having DHA attached to triglycerides; adding any one or mixture of alcoholic sodium hydroxide and potassium hydroxide to the selected fatty acid to form a reaction mixture; stirring the reaction mixture, wherein the reaction mixture separates into two layers (upper and lower layer), discarding the upper layer containing lower fatty acids having less than 20 carbons with the triglycerides and other impurities; adding a ketone to the second mixture; stirring the second mixture; thereafter allowing the DHA to precipitate from the second mixture; filtering the second mixture to separate the precipitate; washing the precipitate with the ketone to remove impurities and other polyunsaturated free fatty acids; evaporating the precipitate at room temperature for recovering the DHA in form of crystalline mass; passing the crystalline mass of DHA through sieve to obtain a dry, directly compressible, free flowing powder of free fatty acid DHA.

### BRIEF DESCRIPTION OF DRAWINGS

FIG. 1 illustrates a flowchart of a method of isolation of docosahexaenoic acid (DHA) in its polyunsaturated free fatty acid form, in accordance with the present invention; and

FIGS. 2-6 illustrates various spectras of the DHA free fatty acid powder, in accordance with the present invention.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

Reference in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment.

The present invention provides Docosahexaenoic Acid (DHA) and method of isolation thereof. The DHA isolated using the method of the present invention is in free flowing powder form. Further, the DHA in powder form is free from triglycerides. The DHA powder of the present invention has purity more than 90% and is stable at room temperature. This DHA offers excellent bioavailability.

In our invention we get the DHA free fatty acid as free flowing powder which is directly compressible. Being powder, it is found to be stable at room temperature and therefore, addition of antioxidants is not required. The method has minimum steps of purification and doesn't require subzero temperature at any stage for isolation of free fatty acid form of DHA.

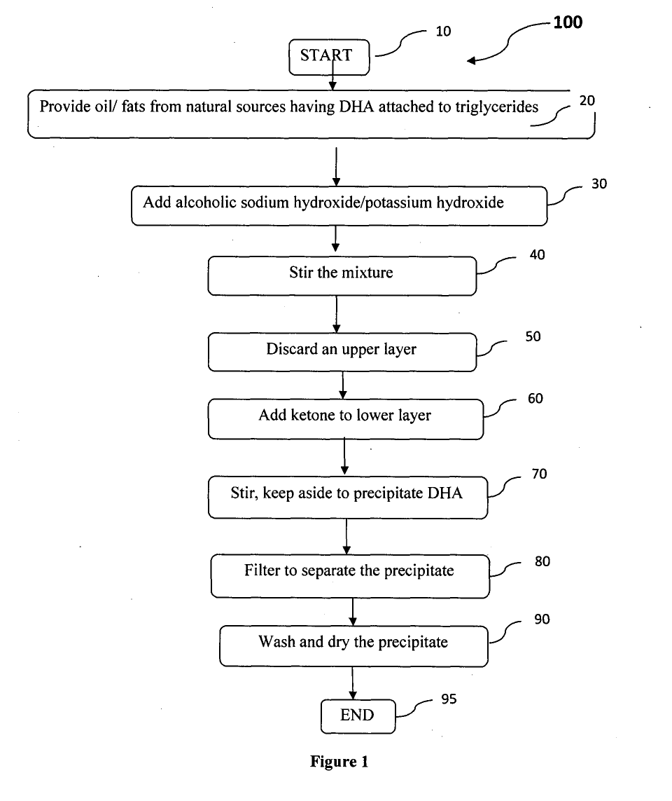

Referring now to FIG. 1, there is shown a flowchart of a method of isolation of the Docosahexaenoic Acid (herein after ‘DHA’). Specifically, the FIG. 1 shows flowchart of the method (100). The method starts at step (10).

At step (20), the method (100) includes providing any one of oils and fats from natural sources having DHA attached to triglycerides. Initiate the method by maintaining the temperature of oil/fat to around 40-45° C.

At step (30), the method (100) includes adding equal quantity of any one or mixture of alcoholic sodium hydroxide and potassium hydroxide step (20) to form a reaction mixture. In an embodiment, the alcoholic solution of 1-2% sodium or potassium hydroxide is selected from methanolic, ethanolic, propanolic, butanolic sodium or potassium hydroxide and combination thereof.

At step (40), the method (100) includes moderately stirring the mixture for up to 30 min. Stirring lower downs temperature of the mixture. After stirring, it separates the mixture into two layers. The upper layer contains the lower fatty acids having less than 20 carbons, the triglycerides and other impurities.

At step (50), the method (100) includes discarding the upper layer.

At step (60), the method includes adding a ketone to the lower layer of the two layers in equal quantity to form a second mixture. In an embodiment, the ketone is selected from acetone, ethyl ketone, methyl ketone and the like.

At step (70), the method (100) includes stirring the second mixture. In an embodiment stirring is carried out for an hour and keep aside the second mixture for 30 minutes to precipitate DHA.

At step (80), the method (100) includes filtering the second mixture to separate the precipitate having DHA.

At step (90), the method (100) includes washing the precipitate several times with the ketone to remove the impurities and other polyunsaturated free fatty acids. Evaporate the ketone and recover it. This leads to drying of the precipitated DHA at room temperature to form the crystalline mass. The same is passed through the sieve to obtain a dry, directly compressible, free flowing powder of polyunsaturated free fatty acid DHA thereof.

In an embodiment, the precipitate is washed with acetone.

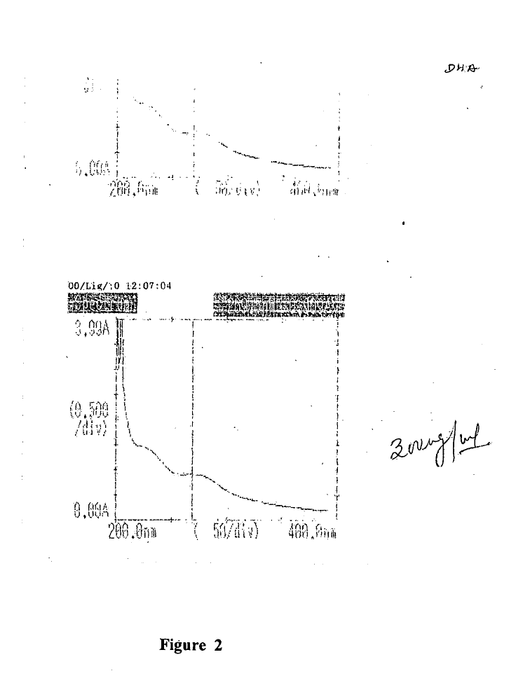

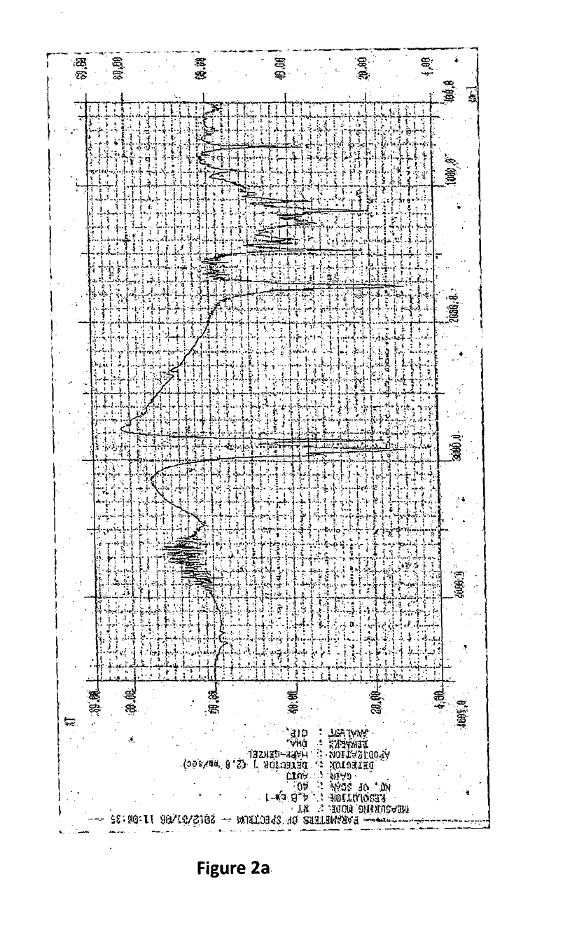

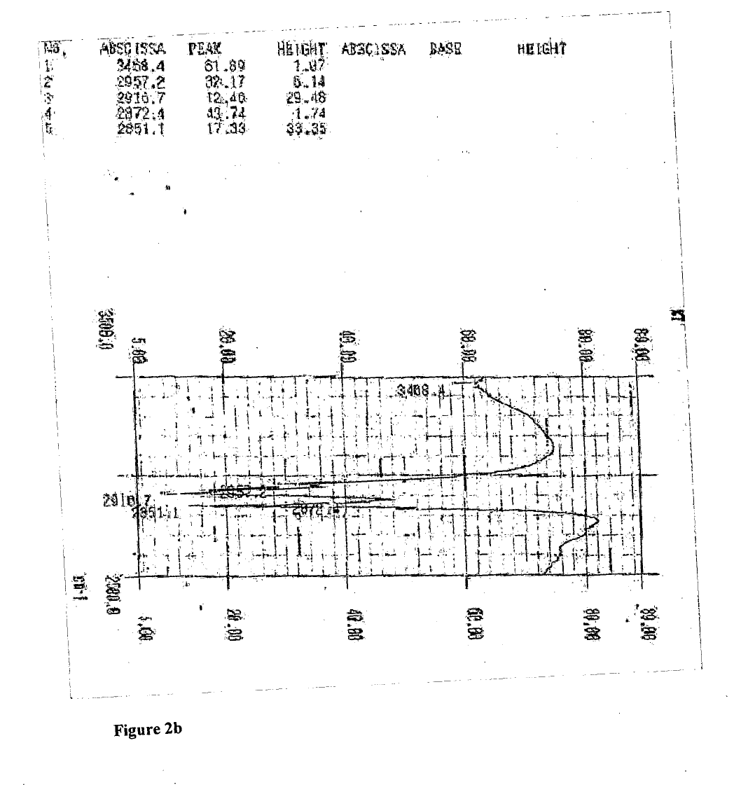

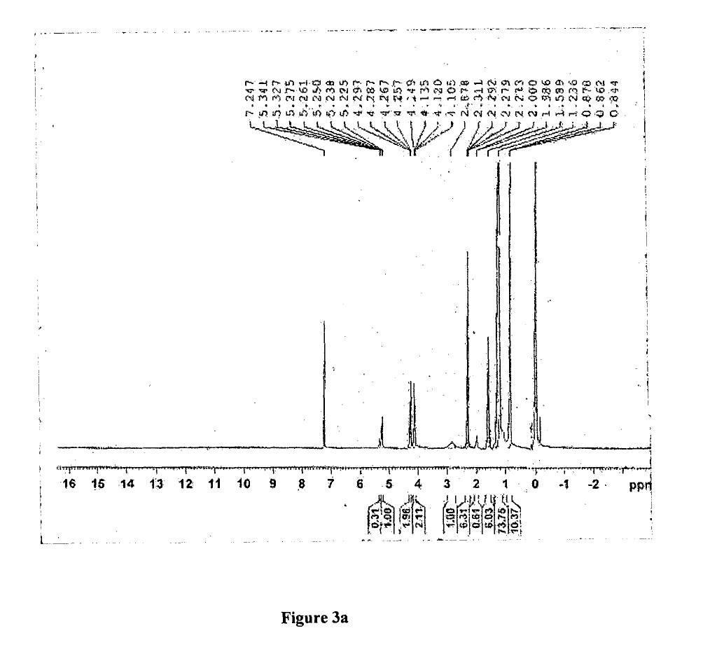

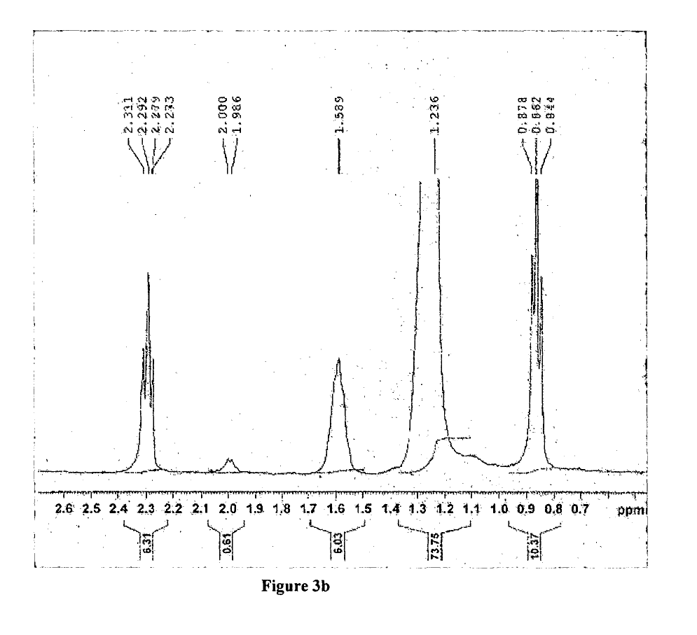

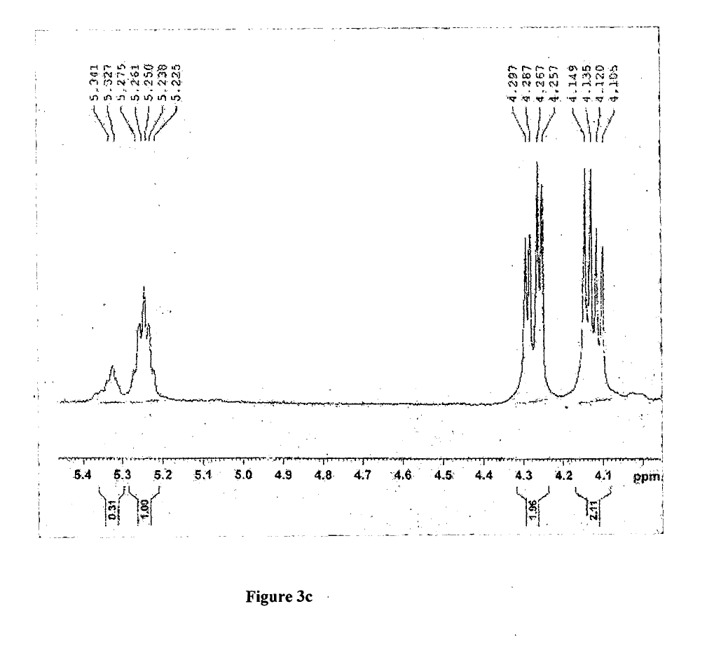

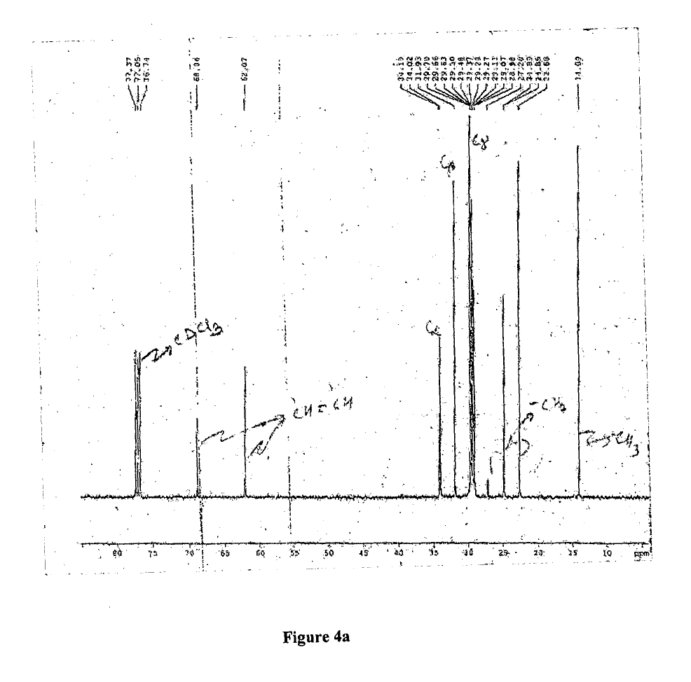

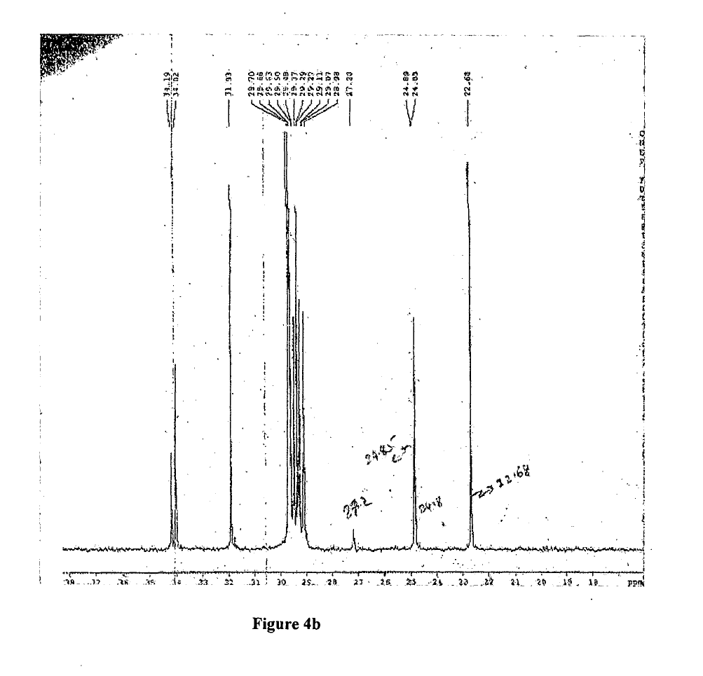

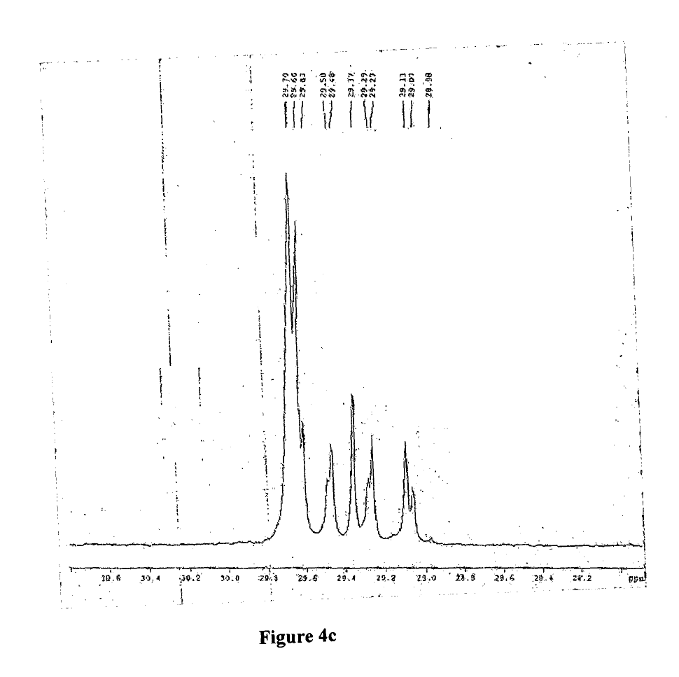

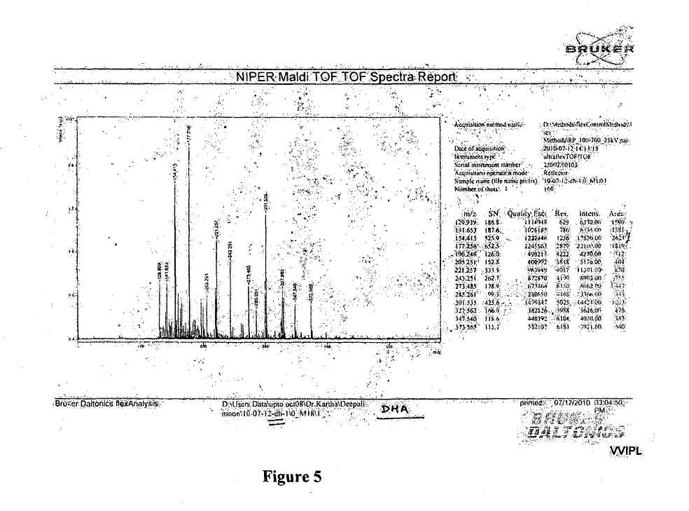

The DHA isolated using the above method is characterized by an UV spectrum (Chart 1, FIG. 2), IR spectrum (Chart 2, FIG. 3a, 3b), H-NMR spectrum (Chart 4, FIG. 4a, 4b, 4c), C13-NMR spectrum (Chart 3, FIG. 5a, 5b, 5c), and Mass spectrum (Chart 5, FIG. 6) substantially similar to FIG. 2-6.

### EXAMPLE 1

Take 1 liter of fish oil. Maintain temperature to 40-45° C. To it add 1-2% methanolic/ethanolic/propanolic/butanolic sodium or potassium hydroxide 1 liter. Stir for ½ an hour. It separates two layers, discard upper layer which contains lower fatty acids and other impurities. Then, to the lower layer add 750 ml of acetone or ethyl or methyl ketone and stir for one hr and keep aside for ½ hour to precipitate DHA. Filter it and wash 2-3 times with equal quantity of acetone. Evaporate and recover the solvent with temperature not exceeding 40 degree C. This leads to drying of the precipitated DHA at room temperature to form the crystalline mass. The same is passed through the sieve to obtain a dry, directly compressible, free flowing powder of polyunsaturated free fatty acid DHA thereof. The DHA is creamish or off-white crystalline solid powder having melting point 60-62 degree C. It is freely soluble in chloroform.

### EXAMPLE 2

Cool fish oil 12 degree C. to solidify all fatty acids in waxy form and separate it from fish oil. Maintain temperature to 40-45° C. In 1 kg waxy fatty acid, add 1 litre ethanolic or methanolic or propanolic or butanolic sodium or potassium hydroxide 1-2% and stir it for ½ an hour. The solution separates the oily layer which contains lower fatty acids and other impurities then, add ethyl acetate:acetone 90:10 to 10:90 to above solvent. It precipitates DHA in solid form. Filter it and give 2-3 wash with above mixture of solvents and evaporate and recover the solvent with temperature not exceeding 40 degree C. This leads to drying of the precipitated DHA at room temperature to form the crystalline mass. The same is passed through the sieve to obtain a dry, directly compressible, free flowing powder of polyunsaturated free fatty acid DHA thereof. The DHA is creamish or off-white crystalline solid powder having melting point 60-62 degree C. It is freely soluble in chloroform.

The spectral analysis for the DHA isolated using the method of the present invention is as follows.

CHART 1
Physical properties of the Isolated DHA and UV

| | | |
|---|---|---|
| M F(probable structure) | = | C₂₂H₃₂O₂ |
| Mol Wt | = | 328 (expected) |
| | = | 327.5 (observed in Mass) |

Physical characterisation of given sample

| | |
|---|---|
| Colour | off-white |
| Odour | fishy smell |
| Texture | waxy |
| Solubility | Chloroform |
| Melting Point | 60-62° C. |

Data given

| | |
|---|---|
| 1. UV/VIS | |
| λ = | 279.6 nm |

This value indicates the presence of unsaturation.

CHART 2
Infrared Spectral Interpretation of DHA
2. IR spectral Interpretation

| Peak Value Observed | | | Expected range | | Inference |
|---|---|---|---|---|---|
| 1. | 3468.4 | cm⁻¹ | (3600-3100 | cm⁻¹) | carboxylic —OH stretching frequency |
| 2. | 2957.2 | cm⁻¹ | (2700-3050 | cm⁻¹) | allphatic region and —CH₃, —CH₂, —CH stretch |
| | 2916.7 | cm⁻¹ | | | |
| | 2851.1 | cm⁻¹ | | | |
| | 2872.4 | cm⁻¹ | | | |
| 3. | 1736 | cm⁻¹ | 1750-1720 | cm⁻¹ | carbonyl stretch |
| 4. | 1637.8 | cm⁻¹ | 1680-1620 | cm⁻¹ | >C═C< stretch |
| | 1647.4 | cm⁻¹ | | | |
| | 1655.1 | cm⁻¹ | | | |
| 5. | 1471.9 | cm⁻¹ | 1475-1300 | cm⁻¹ | CH bending |
| 6. | 895.1 | cm⁻¹ | 1000-650 | cm⁻¹ | aliphatic >C═C< stretch |
| 7. | 717.6 | cm⁻¹ | 990-650 | cm⁻¹ | RCH═CHR stretch |

CHART 3
Nuclear Magnetic Resonance (13c Carbon) Interpretation of DHA
3. ¹³C NMR

| Obsdδ-value (ppm) | Multiplicity pattern | Expectedδ-value | Inference |
|---|---|---|---|
| 173.22 | singlet | 155-185 | >C═O gp (carboxylic region) |
| 77.05, 77.37, 77.74 | triplet | — | CDCl3 solvent |
| 0-85 | — | — | aliphatic region |
| 62.07, 68.86 | singlet | — | —CH═CH— |
| 34.19, 34.02 | doublet | triplet | α to carboxylic gp |
| 31.93 | doublet | triplet | βto carboxylic gp |
| 29.70-29.07 | multiplet | multiplet | δto carboxylic gp |
| 28.98 | singlet | singlet | —CH2 gp |
| 27.20, 24.89, | shoulder | — | respective —CH2 gps |
| 24.85, 72.68 | | | |
| 14.09 | singlet | quartet | —CH3 gp |

CHART 4
Nuclear Magnetic Resonance (1H Proton) Interpretation of DHA

| Observed | Multiplicity | Expected | |
|---|---|---|---|
| δ-value | pattern | δ-value | Inference |
| 0.826 | triplet | 0.9-1.03 | —CH3 |
| 1.208 | multiplet | 2.03 | |
| 1.462 | multiplet | 1.6 | β to carbonyl |
| 1.946 | multiplet | 1.8.1.9 | α to >C═C< |
| 2.067 | multiplet | — | |
| 2.242 | multiplet | 2.3 | α to carbonyl |
| 2.492 | multiplet | 2.6 | 4-CH2 gps(8H) |
| 3.120 | singlet | | |
| 4.075 | multiplet | 5.3-5.4 | —CH═CH—(8H) |
| 5.3 | singlet | 5.5 | C5-C6 protons |
| 7.25 | m | | |
| 7.43 | m | | |
| 7.51 | m | | |
| 8.36-8.4 | singlet | 10-12 | —OH(1H) |

CHART 5
MASS Interpretation of DHA
5. Mass Spectra of DHA

| Obsd m/e value | loss of fragment | loss of group/inference |
|---|---|---|
| 327.5 | — | m.i |
| 279.0 | 45 | COOH |
| 220 | 54(59) | CH₂CH₂CH═CH |
| 199.0 | 21 | —CH═CH— |
| 177.0 | 22(26) | —CH═CH— |
| 154 | 23(26) | —CH═CH— |

On the above observation and interpretation, the structure of the Docosahexaenoic acid (DHA) is found as follows.

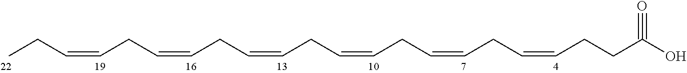

The present invention brings out a pure polyunsaturated free fatty acid DHA in free flowing powder form, which is substantially free from water, directly compressible and it is highly compatible and stable in any form of compositions like solid compositions, liquid compositions, powder compositions, tablets, capsules, gels and all other forms of formulations providing these free fatty acid DHA,

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, to thereby enable others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the present invention.

1. A method of isolation of docosahexaenoic acid (DHA) comprising the steps of:
a) adding at least one alcoholic solution selected from a group comprising alcoholic solution of sodium hydroxide, alcoholic solution of potassium hydroxide, or mixtures thereof to a fatty acid sources to form a reaction mixture;
b) stirring the reaction mixture to obtain an upper layer and a lower layer, wherein said upper layer is discarded;
c) adding a ketone selected from a group comprising of acetone, ethyl ketone, or methyl ketone to the lower layer to form a second mixture;
d) allowing the DHA to precipitate from the second mixture to recover the DHA in a form of crystalline mass,
wherein said crystalline mass of the DHA can be processed to obtain a dry, directly compressible, free flowing powder of free fatty acid DHA;

2. The method of claim 1, wherein the discarded upper layer of step (b) contains lower fatty acids having less than 20 carbons, triglycerides and other impurities.

3. The method of claim 1, wherein the second mixture obtained from step (c) is stirred before allowing DHA to precipitate from said second mixture.

4. The method of claim 1, wherein the second mixture of step (d) is filtered to separate precipitates of DHA.

5. The method of claim 1, wherein the precipitates formed in step (d) are dried at room temperature to recover the DHA in said crystalline mass.

6. The method of claim 1, wherein the precipitates are washed with the ketone to remove impurities and other polyunsaturated free fatty acids.

7. The method of claim 1, wherein the crystalline mass of the DHA is passed through a sieve to obtain said dry, directly compressible, free flowing powder of free fatty acid DHA

8. The method as claimed in claim 1, wherein the fatty acid sources are selected from a group comprising of mackerel oil, menhaden oil, salmon oil, capelin oil, tuna oil, sardine oil, or cod oil, marine algae, or any other source having DHA attached to triglycerides.

9. The method of claim 8, wherein the marine algae is Schizochytrium sp.

10. The method as claimed in claim 1, wherein the alcoholic solution of sodium or the alcoholic solution of potassium hydroxide is selected from a group comprising methanolic, ethanolic, propanolic, butanolic solution or mixtures thereof.

11. A Docosahexaenoic acid (DHA) in a dry, directly compressible, free flowing powder form.
